(19) United States
(12) Patent Application Publication
(10) Pub. No.: US 2025/0360122 A1
(43) Pub. Date: Nov. 27, 2025

(54) USE OF NAMPT INHIBITORS IN THE MANUFACTURE OF MEDICAMENTS FOR PREVENTING AND/OR TREATING PIGMENTED VILLONODULAR SYNOVITIS

(71) Applicant: WEST CHINA HOSPITAL, SICHUAN UNIVERSITY, Chengdu, Sichuan (CN)

(72) Inventors: Weili FU, Chengdu, Sichuan (CN); Minghao GE, Chengdu, Sichuan (CN); Yunan HU, Chengdu, Sichuan (CN); Xin MA, Chengdu, Sichuan (CN); Dingyuan ZHANG, Chengdu, Sichuan (CN); Jian LI, Chengdu, Sichuan (CN)

(21) Appl. No.: 19/294,460

(22) Filed: Aug. 8, 2025

(30) Foreign Application Priority Data
Aug. 8, 2024 (CN) 202411084913.6

(51) Int. Cl. A61K 31/4545; A61P 35/00

(52) U.S. Cl. CPC A61K 31/4545; A61P 35/00

(57) ABSTRACT

The present invention provides the use of NAMPT inhibitors in the manufacture of medicaments for preventing and/or treating pigmented villonodular synovitis (PVNS), and belongs to the field of medicine. In the present invention, based on the PVNS-PDX mouse model, it is found that NAMPT inhibitor FK866 exhibits significant therapeutic effects on PVNS, such as improving clinical symptoms related to the disease and inhibiting the proliferation of PVNS grafts. It is discovered that FK866 can target the NAMPT-ITGA5-JUND signaling axis, alleviate endothelial cell damage and vascular permeability, reduce synovial inflammation, decrease the volume of synovial tumor grafts, mitigate hemorrhagic lesions of PVNS, and hinder tumor progression. Therefore, NAMPT inhibitors have broad application prospects in the manufacture of medicaments for preventing and/or treating PVNS.

### INCORPORATION OF SEQUENCE LISTING

This application contains a sequence listing submitted in Computer Readable Form (CRF). The CRF file contains the sequence listing entitled “4-2-PA2880164-SequenceListing.xml”, which was created on Aug. 7, 2025, and is 16,208 bytes in size. The information in the sequence listing is incorporated herein by reference in its entirety.

### FIELD OF THE INVENTION

The present invention belongs to the field of medicine, and specifically relates to the use of NAMPT inhibitors in the manufacture of medicaments for preventing and/or treating pigmented villonodular synovitis (PVNS).

### BACKGROUND OF THE INVENTION

Pigmented villonodullar synovitis (PVNS) is a benign intra-articular soft tissue tumor, characterized by the proliferative lesions of synovial villus, accompanied by iron-yellow pigment deposition and inflammatory cell infiltration, with local aggressiveness. Clinically, it presents as long-term joint swelling and pain, limited mobility, and bloody joint fluid upon puncture. The incidence rate of PVNS is 4 per million, and it is more common in the knee joint. In addition, PVNS may lead to extra-articular invasion, ultimately resulting in joint replacement or even amputation.

Currently, common treatment methods for PVNS include surgical resection, external irradiation, radioactive synovectomy, and pharmacotherapy, among which arthroscopic resection is the primary treatment method. However, some patients are not suitable for surgery, and even after surgery, the tumor may recur. Therefore, the development of medicaments for treating PVNS is of great significance.

### CONTENT OF THE INVENTION

The objective of the present invention is to provide the use of NAMPT inhibitors in the manufacture of medicaments for preventing and/or treating PVNS.

The present invention provides the use of NAMPT inhibitors in the manufacture of medicaments for preventing and/or treating PVNS.

Further, the NAMPT inhibitor is KF866.

The structure of FK866 is

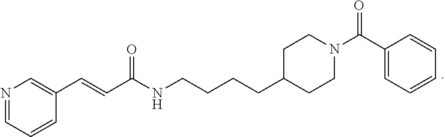 [SMILES: O=C(/C=C/c1cccnc1)NCCCCC1CCN(C(=O)c2ccccc2)CC1]

Further, the medicament is one that alleviates endothelial cell damage.

Further, the medicament is one that alleviates vascular permeability.

Further, the medicament is one that reduces fibrinogen deposition around blood vessels.

Further, the medicament is one that inhibits the growth of synovial tumors.

Further, the medicament is one that alleviates synovial inflammation.

Further, the medicament is a preparation formed with NAMPT inhibitor as the active ingredient, in combination with pharmaceutically acceptable excipients.

Further, the preparation is an oral preparation or an injection preparation.

Further, the oral preparation is tablet, granule, pill, capsule, suspension, or emulsion.

The present invention achieves the following beneficial effects:

The PVNS tissue is composed of macrophages, endothelial cells, vascular smooth muscle cells, fibroblasts, lymphocytes, etc. In patients with PVNS, the infiltration of many immune cells and the increased cytokine secretion in synovial cells lead to inflammation, and the increased cell proliferation and migration result in the manifestation of tumor phenotypes. The present invention reveals an increase in local osteoclastogenesis and macrophage activation in patients with PVNS.

By combined analysis of BulkRNA-seq and proteomics, scRNA-seq and Spatial Transcriptomics, the present invention can identify the NAMPT-ITGA5-JUND signaling pathway as a link between macrophages and endothelial cells, regulating the progression of PVNS disease. NAMPT regulates the differentiation of osteoclast-like macrophages by promoting the role and potential molecular mechanisms of JUND expression in the pathogenesis of PVNS. Therefore, inhibiting the NAMPT-ITGA5-JUND signaling pathway and blocking the damage of osteoclast-like macrophages to endothelial cells holds significant importance in the treatment of PVNS.

In the present invention, it is discovered that FK-866 can inhibit the activation of the NAMPT-ITGA5-JUND pathway in PVNS endothelial cells and macrophages, thereby suppressing abnormal macrophage activation, e.g. inhibiting endothelial cell damage and suppressing the secretion of inflammatory and chemotactic factors by endothelial cells and macrophages. Consequently, it can alleviate synovial inflammation and joint destruction in patients.

Based on the PVNS-PDX mouse model, the present invention reveals that the NAMPT inhibitor FK866 exhibits significant therapeutic effects on PVNS, such as improving clinical symptoms associated with the disease and inhibiting the proliferation of PVNS grafts. It is found that FK866 can target the NAMPT-ITGA5-JUND signaling axis, alleviate endothelial cell damage and vascular permeability, reduce synovial inflammation, decrease the volume of synovial tumor grafts, mitigate hemorrhagic lesions of PVNS, and hinder tumor progression.

Therefore, NAMPT inhibitors have broad application prospects in the manufacture of medicaments for the prevention and/or treatment of PVNS.

Based on the content of the present invention and the common knowledge in this field, it can be inferred that, apart from FK866, other NAMPT inhibitors well-known in this field also have broad application prospects in the manufacture of medicaments for preventing and/or treating PVNS.

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. Pathological processes of PVNS discovered by Bulk RNA-seq and proteomics. Panel A: Schematic diagram of the overall workflow for the research plan according to the present invention. Panel B: Arthroscopic observation and MRI characteristics of PVNS.

FIG. 2. Pathological processes of PVNS discovered by Bulk RNA-seq and proteomics. Panel A: Heatmap for cluster analysis of differential mRNA (TOP40) expression in the control group. Panel B: GSEA enrichment plot of representative signaling pathways, showing upregulated mRNA in the samples of PVNS group compared to the samples of control group. Panel C: Bubble chart of KEGG enrichment analysis (top 10) (upregulated differential mRNA).

FIG. 3. Pathological processes of PVNS discovered by Bulk RNA-seq and proteomics. Panel A: Heatmap for cluster analysis of differential protein (TOP40) expression in the control group. Panel B: GSEA enrichment plot of representative signaling pathways with upregulated protein expression in the samples of PVNS group compared to that of the control group. Panel C: Bubble chart of KEGG enrichment analysis (top10) (upregulated differential proteins).

FIG. 4. Pathological processes of PVNS discovered by Bulk RNA-seq and proteomics. Panel A: Correlation between log2(FC) of differentially expressed mRNAs and log2(FC) of differentially expressed proteins (/log FC/≥1; p-value<0.05), where the red group indicates mRNAs upregulated in RNA-seq and proteins upregulated in proteomics. Panel B: Bubble plot analysis of GO enrichment (TOP20) and co-upregulation of mRNA and protein expression. C: Bubble plot analysis of KEGG enrichment (TOP20) and co-upregulation of mRNA and protein expression genes.

FIG. 5. Pathological processes of PVNS discovered by Bulk RNA-seq and proteomics. Panel A: Principal Component Analysis (PCA) using mRNA expression to illustrate the relationships between samples in different dimensions. Panel B: Principal Component Analysis (PCA) using protein expression to illustrate the relationships between samples in different dimensions.

FIG. 6. Single-cell sequencing analysis of human PVNS synovial cell atlas. Panel A: t-SNE visualization of donor-derived cells in control and PVNS samples. Panel B: t-SNE visualization of all cell clusters in collected specimens. Panel C: Percentage of identified cell clusters in control and PVNS samples. Panel D: t-SNE visualization of cell clusters at each sample level. Panel E: Proportion of cell subsets at each sample level. Panel F: Heatmap of selected marker genes in each cell subpopulation. Panel G: Expression distribution characteristics of selected subpopulation-specific genes.

FIG. 7. Characterization of macrophages. Panel A: t-SNE visualization of macrophage subsets. Panel B: t-SNE visualization of macrophage subset distribution in control and PVNS samples. Panel C: Percentage of identified cell subsets in control and PVNS samples. Panel D: Bubble chart reveals normalized expression of DEGs for each defined subcluster. Panel E: Violin plot shows osteoclast-related genes in macrophage subsets. Panel F: Immunohistochemical staining of ACP5, CTSK, and TNFRSF11A in control and PVNS samples.

FIG. 8. Characterization of endothelial cell subpopulations. Panel A: t-SNE visualization of endothelial cell subpopulations. Panel B: Proportions of seven identified subpopulations in the control group and PVNS group. Panel C: t-SNE visualization of the distribution for endothelial cell subpopulations in control and PVNS samples. Panel D: Percentages of identified cell subpopulations in control and PVNS samples. Panel E: Bubble plot reveals the normalized expression of DEGs for each defined subpopulation.

FIG. 9. Identifying the key role of macrophage subpopulations and characterizing osteoclast-like macrophages in PVNS. Panel A: GO enrichment analysis of differentially expressed genes in macrophage (Macrophages) subpopulations. Panel B: Quantitative immunohistochemical analysis of ACP5, CTSK, and TNFRSF11A. Panel C: GO enrichment analysis of differentially expressed genes in endothelial cell subpopulations.

FIG. 10. Inferred relationship between signal network and vascular endothelial cell injury. Panel A: Comparison network diagram of cell interaction strength in intercellular communication network between tissues. Panel B. Heatmap comparing the number of cell interactions in the intercellular communication network between tissues. Panel C: Circular graph showing potential cytokines and chemokines expressed in immune cells in synovium of PVNS and control groups. Panel D: Bubble chart generated by Cell Chat showing potential ligand-receptor pairs, with close relationship between Mac 6 and endothelial cells. Panel E-G: Violin plots showing representative marker genes of the visfatin signaling pathway expressed in macrophages and endothelial cells. Panel H: Western blot showing the expression levels of NAMPT, ITGA5, and JUND in synovium of different groups. Panel I: SCENIC analysis of endothelial cell subpopulations in control group and PVNS group. Panel J: Immunohistochemical staining of NAMPT, ITGA5, and JUND in control and PVNS samples.

FIG. 11. Single-cell regulatory network inference and clustering (SCENIC) studies the gene regulatory mechanism within endothelial cells of PVNS. Panel A: Quantitative immunohistochemical analysis of NAMPT, ITGA5, and JUND. Panel B: Quantitative Western blot analysis of NAMPT, ITGA5, and JUND. Panel C: Real-time quantitative PCR (n=3 for each group). D: Violin plot showing the expression of ITGB 1 in endothelial cells.

FIG. 12. Results of spatial transcriptomics study on human joint synovium. Panel A: H&E staining of synovium in the PVNS group and control group. Panel B: Spatial heatmaps of ACP5, CTSK, and TNFRSF11A expression in synovial samples. Panel C: Focus plots showing the spatial distribution of different cell populations in the PVNS group and control group. Panel D: Spatial heatmaps of NAMPT, ITGB1, and JUND expression in synovial samples.

FIG. 13. In vivo experimental results of NAMPT inhibitor treatment for PVNS. Panel A: Schematic diagram of the NAMPT inhibitor treatment regimen. Panel B: H&E staining of different groups. Panel C: Western blot showing the expression level of JUND in synovium of different groups. Panel D: JUND immunohistochemical staining results of samples from PDX group and FK886 group (scale bar: 200 μm). Panel E: Evans blue (EB) concentration in PVNS grafts 14 days after FK886 or saline administration; since EB binds to albumin in vivo, this protein leakage into the extravascular space is an indicator of vascular permeability. Values are recorded as EB: ng/mg tissue. Panel F: Immunofluorescence staining of FGB and CD31 in the PDX group and FK886 group (scale bar: 50 μm). Panel G: Image of PVNS synovium on day 16 of grafting.

FIG. 14. In vivo experimental results of NAMPT inhibitor treatment for PVNS. Panel A: In vivo gross image of tumor-bearing kidneys. Panel B: Ex vivo gross image of tumor-bearing kidneys. Panel C: Quantitative analysis of JUND western blot. Panel D: Quantitative analysis of JUND immunohistochemistry.

### EXAMPLES

If the manufacturer of the reagents or instruments used in the present invention is not specified, they are all conventional products that can be purchased through regular channels. Unless otherwise specified, the experimental methods in the examples of the present invention are all conventional methods. Unless otherwise specified, the experimental materials used in the examples of the present invention are all commercially available products.

### Example 1. Genomics and Proteomics Research on Patients with PVNS

### 1. Experimental Method

### (1) Sample Collection

Synovial tissue was collected from 25 patients with PVNS and 15 patients with meniscus injury as controls. The synovial tissue was sourced from patients who had undergone arthroscopic synovectomy. A portion of each sample was frozen in liquid nitrogen and subsequently transferred to a −80° C. refrigerator for further analysis, while the other portion was immediately fixed in 10% neutral buffered formalin. All patients were diagnosed definitively by intraoperative pathological examination and confirmed under arthroscopy.

### (2) Bulk RNA Sequencing

RNA extraction and purification: Synovial tissue (SM) samples were taken from a −80° C. refrigerator and thoroughly ground in a homogenizing tube. Total RNA was extracted using TRIZOL reagent (catalog #15596-018, Life Technologies, Carlsbad, CA, US) according to the manufacturer's instructions, and the integrity of RNA was assessed using an RIN-numbered Agilent Bioanalyzer 2100 (Agilent Technologies, Santa Clara, CA, US). Qualified total RNA was further purified using the RNeasy Micro Kit (#74004, QIAGEN, Hilden, Germany) and RNase-free DNase Set (#79254, QIAGEN, Hilden, Germany). According to the manufacturer's instructions, each slide was hybridized with 1.65 μg of Cy3-labeled cRNA using the Gene Expression Hybridization Kit (catalog #5188-5242, Agilent Technologies, Santa Clara, CA, US) in a hybridization oven (catalog #G2545A, Agilent Technologies, Santa Clara, CA, US). After 17 hours of hybridization, the slides were washed in a staining dish (#121, Thermo Shandon, Waltham, MA, US) using the Genomic DNA Purification Kit (#5188-5327, Agilent Technologies, Santa Clara, CA, US) according to the manufacturer's instructions. Data acquisition: Slide scanning was performed using an Agilent microarray scanner (#G2565CA, Agilent Technologies, Santa Clara, CA, US) with the following default settings: dye channel set to green, scanning resolution at 3 μm, photomultiplier tube (PMT) at 100%, and bit depth at 20 bits. Data extraction was conducted using feature extraction software version 10.7 (Agilent Technologies, Santa Clara, CA, US). The raw data were normalized using the quantile algorithm in GeneSpring software version 11.0 (Agilent Technologies, Santa Clara, CA, US).

### (3) Proteomic Analysis

the SM sample was removed from a −80° C. refrigerator and thoroughly ground in a homogenization tube. To prevent protein degradation, 250 μl of PBS and protease inhibitor (Roche, 4693132001) were added, followed by adding 250 μl of ST buffer (2% SDS+100 mmol/L) for protein denaturation. The test tube was centrifuged at 8000 g for 1 min, and then the supernatant was transferred to a new vial. After boiling water bath and ultrasonic treatment, the supernatant was further centrifuged at 8000 g for 15 min. The resulting supernatant was collected, and the protein concentration was measured using a bicinchoninic acid (BCA) kit (Thermo Fisher, 23235). The protein solution was reduced and alkylated by adding dithiothreitol and iodoacetamide. Then, the solution was replaced with 50 mmol/L ammonium bicarbonate solution by using a FASP chromatography column (PALL, OD010C34). The processed protein solution was digested with trypsin at 37° C. overnight, and then peptides were collected and quantified using a peptide quantification kit (ThermoFisher, 23275). High-performance liquid chromatography (HPLC) conditions and mass spectrometry (MS): Analysis was performed using an EASY-nLC 1200 coupled to an Orbitrap Fusion Lumos Tribrid mass spectrometer system. Peptides were loaded onto a 75 μm×15 cm C18 chromatographic column packed with 1.6 μm C18 particles at a flow rate of 300 nL/min, and separated using a gradient of 0.1% formic acid (solvent A) and 80% acetonitrile+0.1% formic acid (solvent B) over a period of 78 min. The LC separation gradient settings are as follows: 3-8% solvent B in 3 minutes, 8-22% solvent B in 44 minutes, 22-35% solvent B in 16 minutes, 35-55% solvent B in 6 minutes, 55-95% solvent B in 2 minutes, and then maintaining at 95% solvent B for 7 minutes. The temperature of the analytical column is maintained at 55° C. using a column oven kit. The ion source settings, including spray voltage, purge gas, and ion transfer tube temperature, were derived from tuning settings and adjusted according to the current state of the instrument. Specific parameters for the DIA-MS method included 1 MS1 scan and 40 MS2 scans, with an overlap of 1 m/z between windows. For MS1 scans, the scanning range was 350-1500 m/z, with a resolution of 60,000 at m/z 200, an AGC target value set at 1.0×10\hat{ }6, and a maximum injection time of 50 ms. For MS2 scans, the normalized HCD collision energy was set at 32%, with a step size of ±5%, a resolution of 30,000 at m/z 200, an AGC target value of 5.0×10\hat{ }4, and a maximum injection time of 54 ms. Both MS1 and MS2 scans adopt the same settings: the RF lens was set at 40%, and spectra were collected in profile mode.

### (4) Differential Gene Screening

The raw data were normalized using Gene Spring software, and then fold change (the fold of expression difference) and Student's t-test were applied as statistical methods for screening differentially expressed genes. The selection criteria were as follows: 1. Fold change (linear)<=2 or fold change (linear)>=2; 2. T-test p-value<0.05.

### (5) Processing of Single-Cell RNA-seq Data

Using CellRanger count version 4.0.0, the sequencing data was aligned with the human reference genome GRCh38 to obtain gene expression matrices at the single-cell level. Subsequently, the gene expression matrices were imported into a Seurat object for further downstream analysis (version 4.3.0). To ensure the quality of cells used in subsequent analysis, a rigorous quality control (QC) pipeline was implemented. Specifically, for each sample, DoubletFinder was used to predict droplets that might encapsulate multiple cells, and only cells predicted as “Singlet” were retained. Cells exhibiting a high proportion of mitochondrial gene expression (>10%) and a high proportion of transcripts mapped to dissociation-inducing genes (>10%) were filtered out by referencing previous studies. Additionally, cells with unique molecular identifier (UMI) counts (<500), gene counts (<200), and log10GenesPerUMI values less than 0.8 were also excluded.

### (6) Single-Cell RNA-seq Data Analysis

After filtering, 52341 cells were obtained for subsequent analysis in the present invention. SCTransform normalization was applied, followed by PCA dimensionality reduction using the top 3,000 variable genes excluding mitochondrial, ribosomal, and hemoglobin genes. Harmony was used to eliminate batch effects. FindNeighbors selected the top 9 PCs to calculate the KNN nearest neighbor distance, followed by FindClusters for unsupervised clustering. Nonlinear dimensionality reduction was performed using t-SNE or UMAP for single-cell data visualization, consistent with unsupervised clustering. A resolution of 0.7 was selected for both clustering and nonlinear dimensionality reduction. To identify specific cell types, the FindAllMarkers function was applied to precisely locate marker genes from clusters derived from unsupervised clustering. To delve deeper into the degeneration process of immune cells on synovial cells, specific cell subpopulations were extracted for more in-depth analysis. This involved repeating the steps of SCTransform normalization, dimensionality reduction, batch effect removal, clustering, and marker gene identification.

### (7) Differential Gene Expression Analysis

Differential gene expression analysis was performed using the FindAllMarkers or FindMarkers function in Seurat, to compare the differential gene expression between control individuals and PVNS patients, as well as between cell type subcluster markers. The default criteria included a log-transformed fold change greater than 0.25, a corrected P-value less than 0.05, and gene expression in more than 10% of the cells. These analyses were carried out with MAST, which employed a specialized barrier model tailored for scRNA-seq data, unless otherwise stated.

### (8) Transcription Factor Regulon Analysis

To predict the potential transcriptional regulatory network in FSPC, SCENIC analysis was performed using pySCENIC in the present invention. The input matrix for FSPC was the normalized expression matrix obtained from Seurat. The AUCell module of pySCENIC was used to evaluate the regulon activity in AUC units, with a default threshold. The Wilcoxon rank-sum test within the FindAllMarkers function of Seurat was utilized to identify differential expression of regulons.

### (9) Analysis of Cell-Cell Interactions

Using Cell Chat (v1.6.1), intercellular interactions were inferred based on the expression of known ligand-receptor pairs in different cell types. The present invention followed the official tutorial to identify potential intercellular communication networks between macrophages and endothelial cells in PVNS. Specifically, the present invention loaded normalized counts of relevant cell groups into CellChat, and applied preprocessing functions based on standard parameter settings, including identifying overexpressed genes, identifying overexpressed interactions, and project data. In the preliminary analysis, the core function computeCommunProb was used together with the population of parameter sets. size=TRUE, assuming that abundant cell populations tended to transmit stronger signals collectively than scarce cell populations. Additionally, standard parameters were applied in the calculation of CommunProb Pathway and aggregateNet. Finally, to identify significant signal changes between Control and PVNS, the present invention utilized various comparison and visualization functions in Cell Chat, such as comparing interactions, netVisual_heatmap, and ranking similarity.

### (10) Functional Analysis

The present invention underwent gene set enrichment analysis, including the enrichment of GO and GSEA in Reactome using the clusterProfiler package (version 3.18.1).

### (11) Processing of Spatial Transcriptomics Data

The raw sequencing reads from spatial transcriptomics underwent quality control and were mapped to the pre-constructed human reference genome GRCh38 using Space Ranger v2.0.1 with default settings. The gene spot matrix from Space Ranger was imported into a Seurat object for further quality control and analysis using the Seurat software package (version 4.3.0). In the present invention, spots were filtered based on the detection of at least 200 genes and a minimum of 500 UMI counts, while removing genes expressed by fewer than 10 spots. Additionally, spots with mitochondrial counts of >10% or ribosomal counts of >20% were excluded.

### (12) Analysis of Spatial Transcriptomics Data

The present invention applied SCTransform normalization at the filtering points and utilized the first 11 principal components (PCs) from principal component analysis (PCA) to perform clustering at a resolution of 0.6. Cells were visualized in the same dimensions used in clustering using the Uniform Manifold Approxation and Projection (UMAP) algorithm. The spatial feature expression plots were generated using the SpatialFeaturePlot and VInPlot functions in Seurat (version 4.3.0). To assign cell types to synovial samples in spatial transcriptomics, the present invention employed Robust Cell Type Decomposition (RCTD) in full mode. This method allowed for the assignment of multiple cell types at each point and was particularly recommended for platforms such as 10× Genomics Visium. The vizAllTopics function in STdeconvolve (version 1.6.0) was used to visualize the proportional weight of each cell type within a single point.

### (13) iTALK Analysis

In the present invention, the iTALK package was also utilized to reveal the intercellular regulation of cytokine and chemokine levels in normal and disease groups, respectively. The original count matrices of Mac6 and Ec2 were inputted into the present invention. Only the top 100 regulatory effects were selected and displayed. In the present invention, if the ligand originated from a cytokine, this regulatory effect was marked with a red line, while other regulatory effects were marked with a black line.

### 2. Experimental Results

### (1) Patients and Clinical Samples

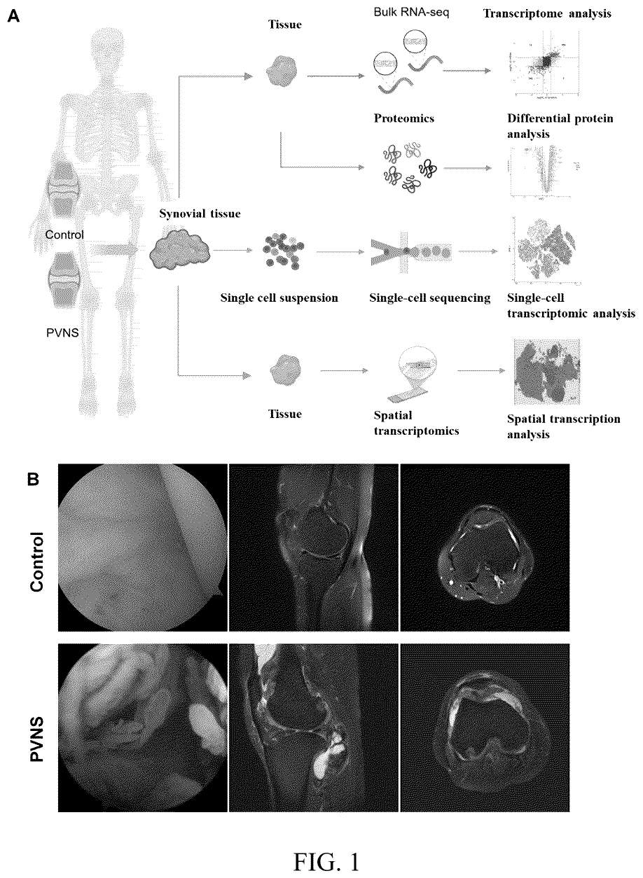

In the present invention, synovial tissue samples obtained from four patients diagnosed with PVNS and four matched adjacent normal tissues were comprehensively analyzed, including extensive RNA-seq data, proteomics data, spatial transcriptomics analysis, and scRNA-seq. For this study, 25 patients diagnosed with PVNS of the knee joint and 15 patients with meniscus injury of the knee joint were selected as the sources of synovial samples, which were designated as the PVNS group (P) and the Control group (C), respectively. In this invention, the general characteristics of the PVNS group was carefully examined using arthroscopy and MRI. Arthroscopic images vividly depicted the synovial tissue of patients in the PVNS group, exhibiting a unique reddish-brown hue due to hemosiderin deposition, with a pronounced proliferative morphology compared to the Control group (FIG. 1 Panel B). Meanwhile, MRI scans showed high signal intensity of the synovial tissue in the fs-pd sequence. Notably, patients with PVNS exhibited significant joint swelling and cartilage erosion within the knee joint (FIG. 1 Panel B).

### (2) Pathological Processes Revealed by Bulk RNA-seq and Proteomics

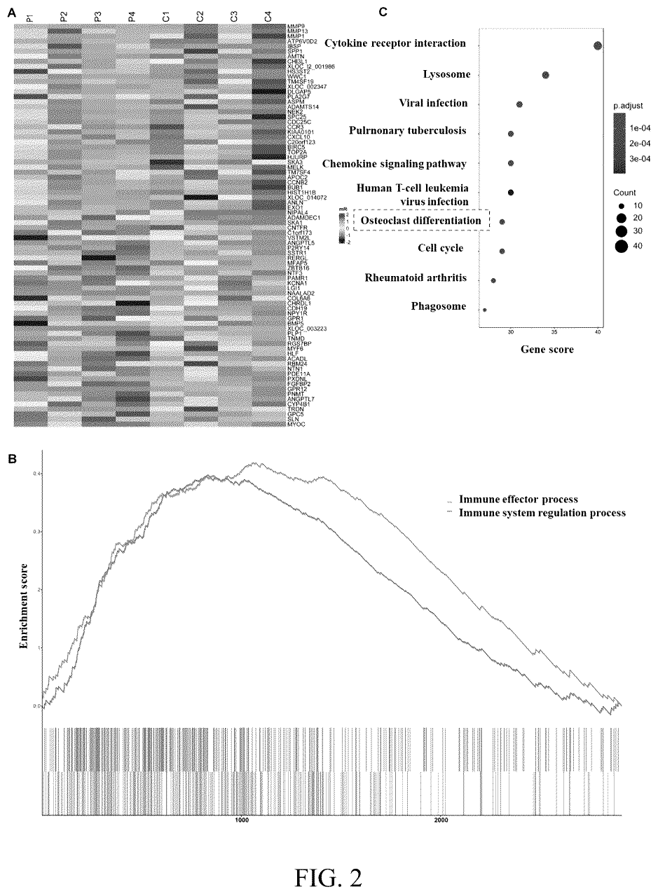

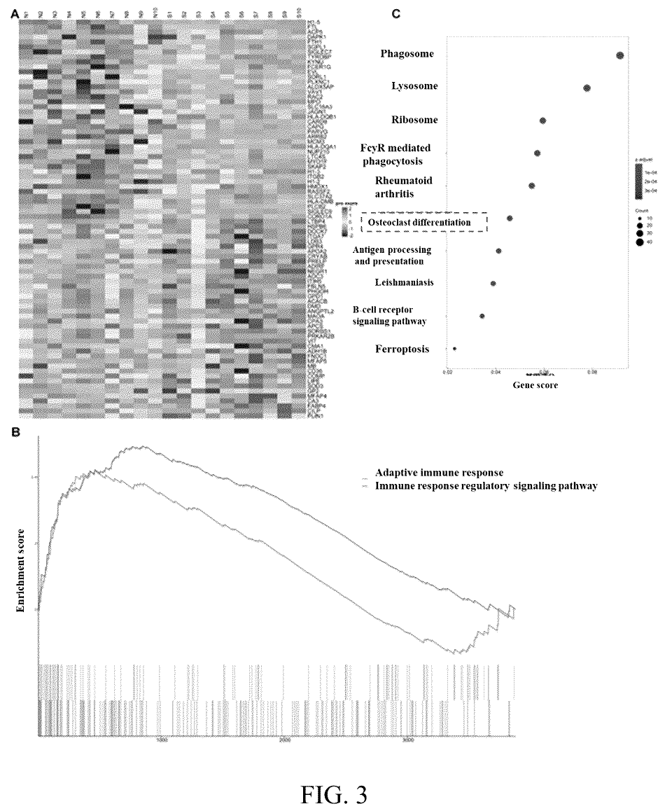

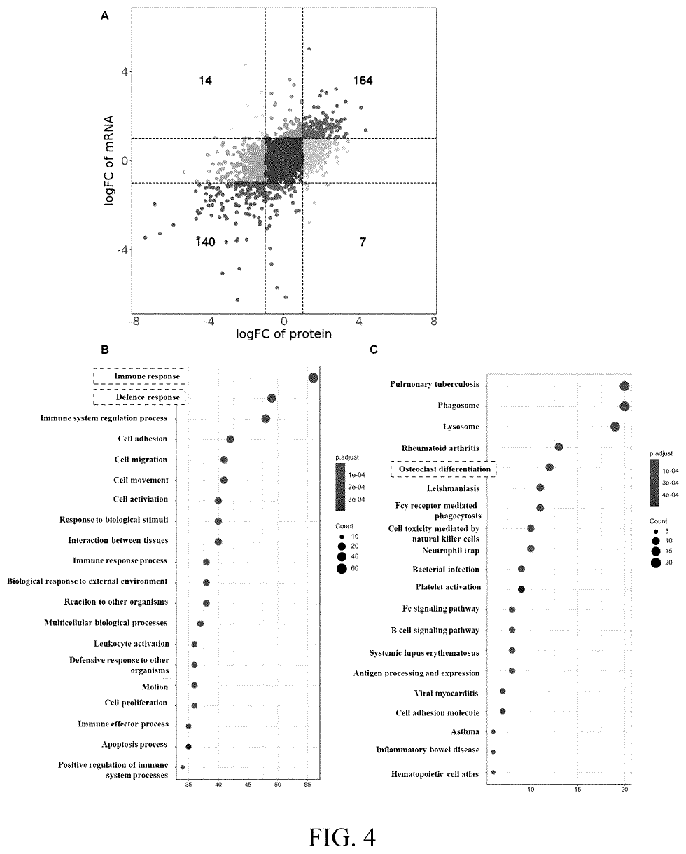

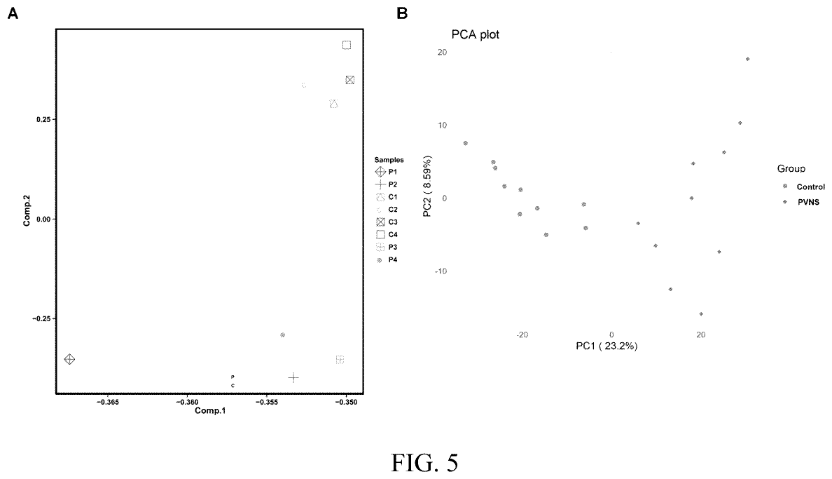

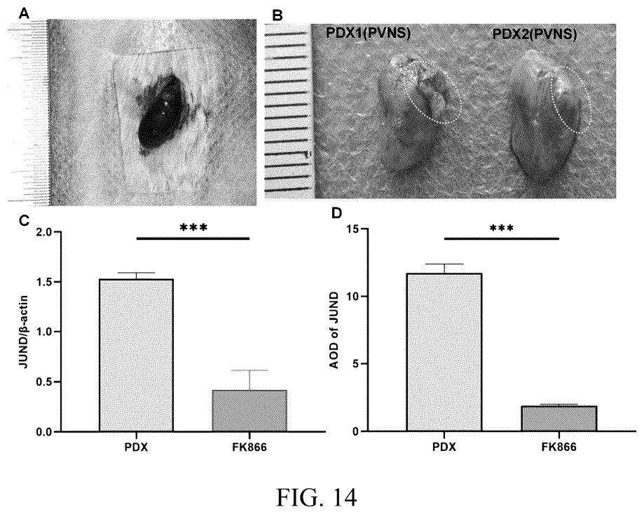

Principal Component Analysis (PCA) visualization revealed that the samples from the same group tended to cluster together, indicating a more compact spatial arrangement, while samples from different groups exhibited a greater degree of dispersion (FIG. 5 Panel A, Panel B). Batch RNA-seq analysis uncovered a total of 1785 differentially expressed mRNA genes, including 994 upregulated genes and 791 downregulated genes. The heatmap of the top 40 differentially expressed genes provided further clarification, revealing enhanced expression of genes related to inflammation and injury in the PVNS group compared to the Control group (FIG. 2 Panel A). Subsequently, Gene Ontology (GO) and Gene Set Enrichment Analysis (GSEA) of Differentially Expressed Genes (DEGs) revealed enrichment in processes related to immune factors and immune system regulation (FIG. 2 Panel B). Additionally, Kyoto Encyclopedia of Genes and Genomes (KEGG) enrichment analysis indicated that osteoclast differentiation was a significantly affected pathway in PVNS (FIG. 2 Panel C). In proteomic analysis, the heatmap of the top 40 differentially expressed proteins showed that, compared to the Control group, the expression of proteins related to inflammation and injury increased in the PVNS group (FIG. 3 Panel A). The GO and GSEA enrichment analysis of DEGs indicated their involvement in adaptive immune response and regulation of immune response (FIG. 3 Panel B). Further analysis of KEGG enrichment for differentially expressed proteins revealed pathways such as antigen processing and presentation, as well as osteoclast differentiation (FIG. 3 Panel C). Joint analysis of differentially expressed mRNA and proteins revealed distinct patterns, with particular emphasis on 164 upregulated genes and proteins in the third quadrant (FIG. 4 Panel A). The GO enrichment analysis of these genes highlighted functions related to immune response and immune system regulation (FIG. 4 Panel B), while the KEGG enrichment analysis indicated osteoclast differentiation (FIG. 14 Panel C). Overall, the differentially expressed genes and proteins identified by extensive RNA-seq and proteomic analyses were enriched in processes related to immune regulation, monocyte differentiation, and osteoclast differentiation, providing valuable insights into the pathogenesis of PVNS.

### (3) Single-Cell Sequencing Analysis of the Cell Atlas of PVNS in Human Knee Joints

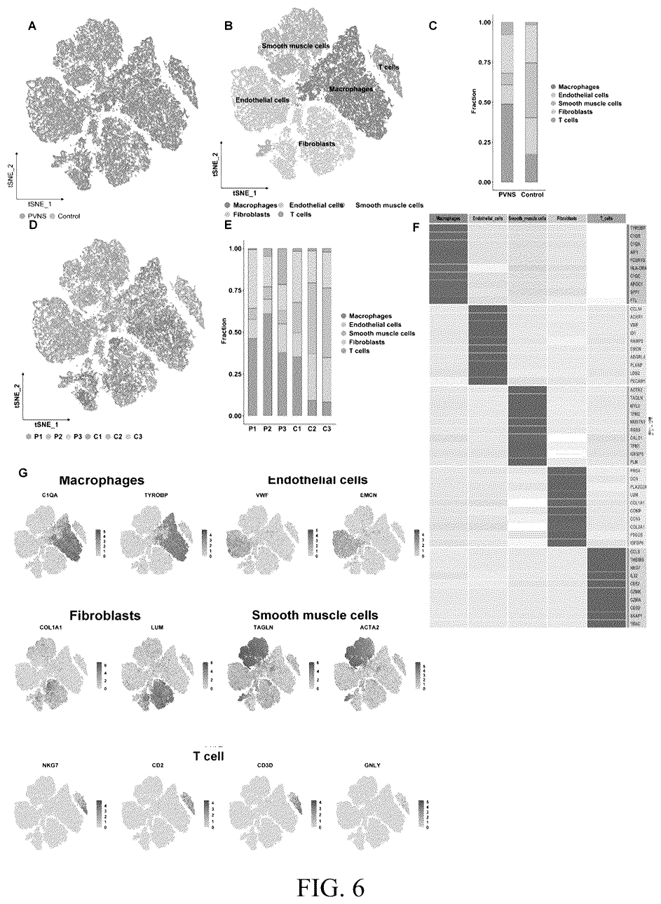

To delve deeper into the differentiation mechanisms of monocytes and osteoclasts, single-cell RNA sequencing analysis was performed in this invention. Overall, this invention identified five distinct cell clusters. Initially, unsupervised t-distributed Stochastic Neighbor Embedding (t-SNE) was employed to depict the cellular composition of all samples (FIG. 6 Panel A). Subsequently, five major cell types were identified in this invention, namely macrophages, fibroblasts, endothelial cells, vascular smooth muscle cells, and T cells (FIG. 6 Panel B, Panel C), distinguished based on specific marker genes (FIG. 6 Panel F). Specifically, macrophages were described by marker genes C1QA and TYROBP, endothelial cells by VWF and EMCN, vascular smooth muscle cells by TAGLN and ACTA2, fibroblasts by COL1A1 and LUM, and T cells by CD2, CD3D, NKG7, and GNLY (FIG. 6 Panel G). Notably, different distributions of cells from different specimens were observed on the t-SNE map, especially in macrophages, with significant differences between the Control group and the PVNS group (FIG. 6 Panel D). Subsequently, the cellular composition of each sample was meticulously examined in the present invention. Compared to the Control group, the increase in macrophages was most significant, becoming the predominant cell type in the PVNS group. Conversely, the number of vascular endothelial cells associated with blood vessels decreased in the PVNS group. Furthermore, a reduction in the number of vascular smooth muscle cells was also observed in the PVNS group (FIG. 6 Panel E).

### (4) Identifying the Key Role of Macrophage Subpopulations and Characterizing Osteoclast-Like Macrophages in PVNS

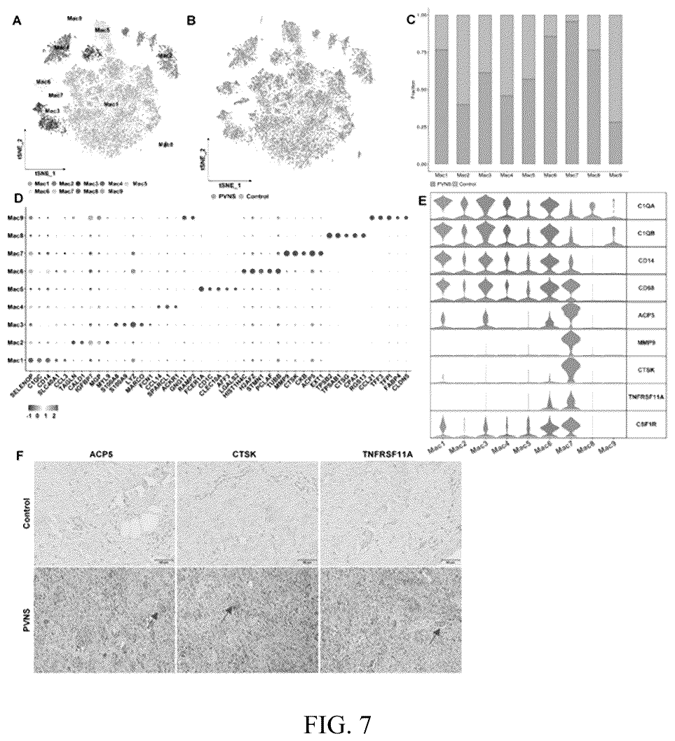

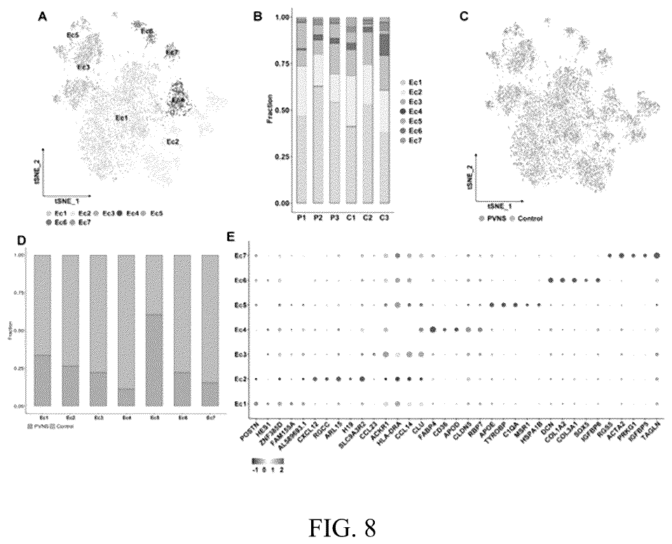

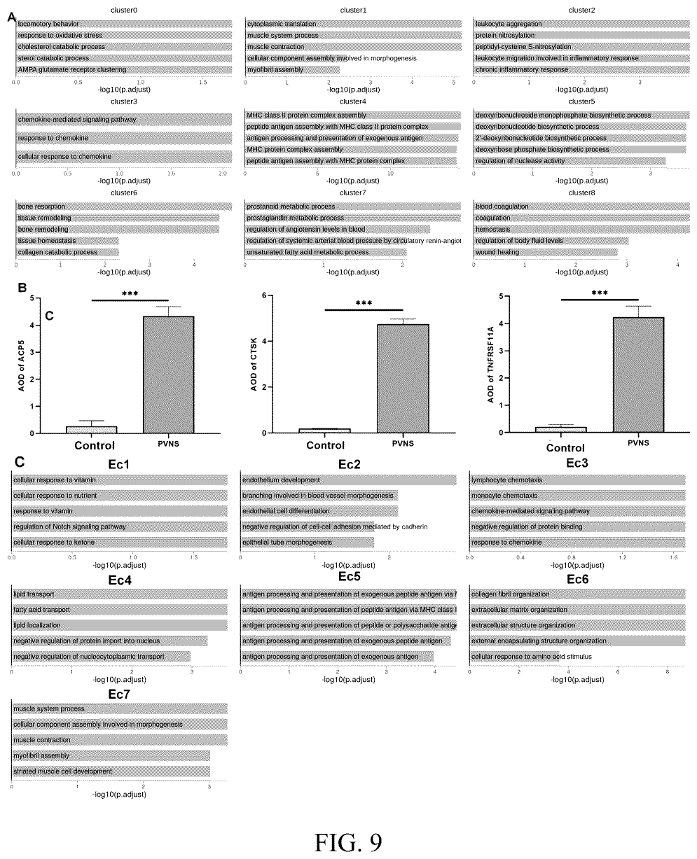

Utilizing insights into monocyte and osteoclast differentiation obtained from extensive RNA-seq and proteomic analyses, a comprehensive clustering analysis of the macrophage populations across all samples was carried out in this invention, revealing nine distinct macrophage subpopulations (FIG. 7 Panel A). These nine subpopulations were named Mac1, Mac2, Mac3, Mac4, Mac5, Mac6, Mac7, Mac8, and Mac9. Visualization of the macrophage clusters on a t-SNE plot revealed distinct distributions, with varying proportions of each subpopulation observed across different sample groups. Notably, all macrophage subpopulations exhibited higher proportions in the PVNS group compared to the Control group, with Mac6 and Mac7 showing the most significant differences (FIG. 7 Panel B, Panel C). To characterize these macrophage subpopulations, DEG was performed across these nine clusters in this invention (FIG. 7 Panel D). Subsequently, by integrating observed changes in cell cluster proportions and differential gene expression, this invention delineated the distinct functional characteristics of each macrophage subpopulation by GO enrichment analysis (FIG. 9 Panel A): Mac1 was identified as a basal macrophage subpopulation, exhibiting responses to chemokines and chemotactic cells (C1QC, CD14); Mac2 was associated with GO terms related to muscle contraction and muscular system processes, defining a subset of chemotactic motile cells (CALD1, TAGLN); Mac3 was characterized by responses to tumor necrosis factor, representing a precursor subpopulation of macrophage differentiation (S100A9, S100A9); Mac4 was linked to chemokine-mediated signaling pathways, indicating a subpopulation of cells functionally related to immunochemical chemotaxis (ACKR1, SPARCL1); Mac5, enriched with GO terms related to antigen processing and exogenous antigen presentation, delineated an antigen presentation-related subpopulation (CD1C, HLA-DQB1); Mac6 exhibited GO terms related to regulation of intrinsic apoptotic signaling pathways, defining it as an invasion-related subpopulation (H2AFZ, STMN1); Mac7 displayed GO terms related to bone remodeling, representing a functional subpopulation involved in matrix remodeling; Mac8 was characterized by GO terms related to prostaglandin metabolic processes, defining a subset with inflammatory regulatory functions (TPSB2, TPSAB1). Finally, Mac9 exhibited GO terms related to blood coagulation, defining it as a subgroup of tissue repair functions involved in wound healing (TFF3, TFPI). Comparative analysis of DEGs between the Mac6 and Mac7 groups (FIG. 7 Panel E) revealed upregulation of osteoclast-related genes, including ACP5, CTSK, and TNFRSF11A. Subsequent examination of the gene expression profiles of Mac6 and Mac7 cells between groups indicated that these subpopulations were primarily responsible for pathological damage and tissue destruction in PVNS, thus confirming the designation of osteoclast-like macrophage clusters. Immunohistochemical (IHC) analysis further confirmed the presence of osteoclast-like macrophages in PVNS tissues (FIG. 7 Panel F, FIG. 9 Panel B). Due to the increased vascular permeability and subsequent blood leakage associated with this condition, there was significant accumulation of red blood cells in the synovial fluid. Cluster analysis of endothelial cell subpopulations identified seven distinct endothelial cell subpopulations (FIG. 8 Panel A), namely Ec1, Ec2, Ec3, Ec4, Ec5, Ec6, and Ec7. Compared to the Control group, the proportions of all six cell subpopulations in the PVNS group decreased, except for Ec5 (FIG. 8 Panel B). Additionally, significant differences were observed in the proportions of endothelial cell subpopulations between each sample in the PVNS and Control groups (FIG. 8 Panel C, Panel D). In-depth analysis of DEGs across these seven subpopulations was conducted (FIG. 8 Panel E), and further GO enrichment analysis of these genes was supplemented (FIG. 9 Panel C). The GO terms for the Ec1 group described it as a subgroup of basal endothelial cells involved in cellular response to nutrients (POSTN, HES1). The Ec2 group was characterized as an injury-induced subgroup of endothelial cells (RGCC, CXCL12), with GO terms including negative regulation of cadherins and cell-cell adhesion mediated by endothelial development. Meanwhile, the Ec3 group was described as an immune chemotaxis-related subgroup (ACKR1, CCL23), with GO terms such as monocyte chemotaxis and chemokine-mediated signaling pathways. On the other hand, the Ec4 group represented lipid-bound endothelial cell subpopulations associated with GO terms such as lipid transport and fatty acid transport (FABP4, CD36). The Ec5 group was identified as an immune response subpopulation involved in antigen processing and exogenous peptide antigen presentation (APOE, TYROBP). Additionally, the Ec6 group was defined as a vascular extracellular matrix-related subpopulation (DCN, COL1A2), with GO terms including collagen fiber tissue and extracellular matrix tissue. The GO terms attributed to the Ec7 group included “muscle system process” and “assembly of cellular components involved in morphogenesis”, defining it as an epithelial-mesenchymal transition subpopulation (RGS5, ACTA2). The present invention assumed that the Ec2 group was significantly associated with endothelial cell damage.

### (5) The Inferred Signaling Network was Associated with Vascular Endothelial Cell Damage

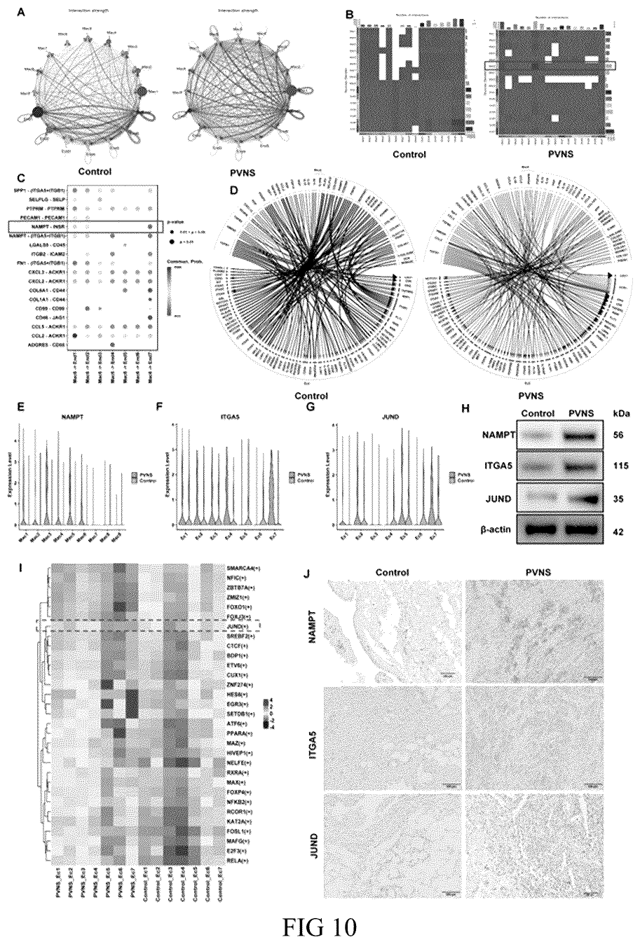

To elucidate the interactions between macrophage and endothelial cell subpopulations in the PVNS microenvironment, and to clarify the underlying mechanisms of PVNS-related inflammation and infiltration damage, CellChat analysis was employed in this invention. The chord diagram (FIG. 10 Panel A) indicated that the PVNS group exhibited stronger intercellular interaction intensity compared to the Control group. Enhanced crosstalk was primarily observed between Mac6 and Ec2, Ec4, Ec7, and other major cell clusters. The interaction number heatmap of the present invention further demonstrated (FIG. 10 Panel B) that, compared to the Control group, the Mac6 subpopulation showed the most significant increase in communication with other cell subpopulations in the PVNS group.

Clinical observations revealed significant blood infiltration and red blood cell leakage in the joints of patients with PVNS, indicating potential damage to the vascular endothelial cells in the affected areas, which was closely correlated with disease progression. The ring diagram of the representative ligand-receptor interaction also demonstrated that, compared to the normal group, the interactions of cytokines and chemokines, such as CCL2, CCL3, CCL4, CCL5, and CCL3L1, were significantly increased in the PVNS group (FIG. 10 Panel D). It could be inferred that the inflammatory level in PVNS was significantly higher than that in normal specimens, thereby promoting damage to endothelial cells by macrophages. These findings suggested that the inflammatory level in PVNS tissues was significantly elevated compared to Control samples, thus facilitating the progression of hemorrhagic lesions in the joints. The bubble chart of the signal pathway relationship according to the present invention (FIGS. 10 Panel C, Panel E, Panel F) showed that in the PVNS group, the NAMPT expressed by Mac6 essentially activated the ITGA5/ITGB1 receptors on Ec2 and Ec7. This ligand-receptor pair was a component of the Visfatin signaling pathway, primarily involved in inflammation and immune responses, and served as a therapeutic target for inflammatory diseases. The effect of NAMPT on endothelial cells lead to endothelial cell damage and vascular structural disruption, thereby accelerating disease progression and exacerbating clinical symptoms.

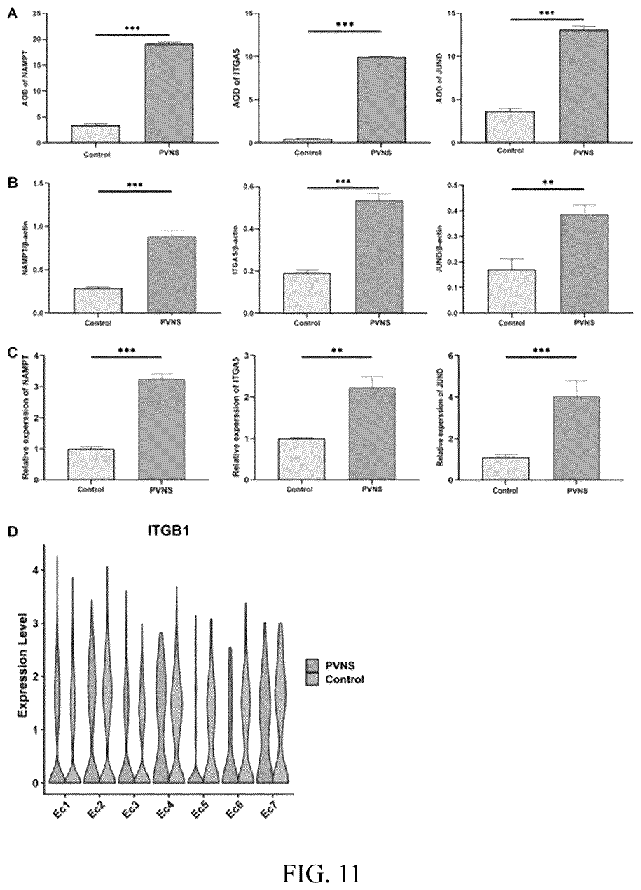

Subsequently, Single-Cell Regulatory Network Inference and Clustering (SCENIC) was performed in the present invention, to further investigate the gene regulatory mechanism within PVNS endothelial cells. Notably, the PVNS group exhibited higher JUND levels (FIG. 10 Panel G, Panel I). Previous studies had shown that NAMPT indirectly activated JUND, leading to articular cartilage damage. The present invention further verified the protein expression levels of NAMPT, ITGA5, and JUND in both groups, confirming the scRNA-seq findings of the present invention (FIG. 10 Panel H, Panel J, and FIG. 11 Panel A, Panel B). It was worth noting that there was no significant difference in ITGB1 expression between the two groups (FIG. 11 Panel D). mRNA expression analysis also indicated consistency with the protein expression results of NAMPT, ITGA5, and JUND obtained in the present invention (FIG. 11 Panel C). In conclusion, the research findings of the present invention indicated that macrophages regulated the expression of the transcription factor JUND in endothelial cells through the Visfatin signaling pathway, thereby exacerbating endothelial cell damage. This mechanism might contribute to the inflammatory environment and vascular destruction observed in PVNS, highlighting potential targets for therapeutic intervention.

### (6) Spatial Transcriptomics of Human Joint Synovium

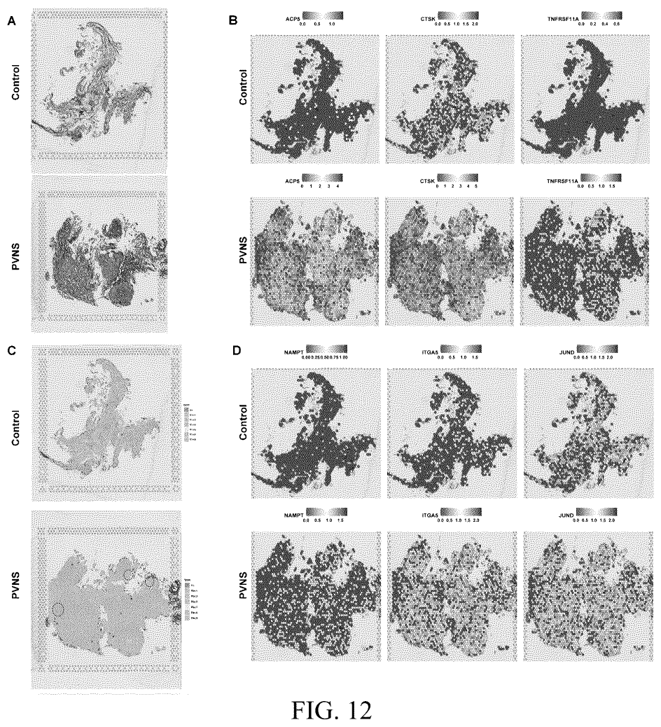

To gain a more comprehensive understanding of the molecular mechanisms driving the progression of PVNS, the spatial transcriptomic sequencing on samples from the PVNS and Control groups was carried out in this invention. Hematoxylin and eosin (H&E) staining revealed disorganized cell arrangement and fibrotic areas in the diseased tissues of the PVNS group, distinguishing it from the Control group (FIG. 12 Panel A). Subsequently, this invention confirmed the presence of osteoclast-like macrophages in the spatial transcriptomics (ST) data by examining the expression of ACP5, CTSK, and TNFRSF11A. Notably, compared to the Control group, the expression levels of these osteoclast-like macrophage marker genes were significantly increased in the PVNS group (FIG. 12 Panel B). In addition, by deconvolution analysis, in this invention, the scRNA-seq data of macrophages and endothelial cells were spatially mapped onto transcriptomic data. In the PVNS group, compared to the Control group, a more complex cellular composition was observed, with an increased abundance of Mac6 cells. Furthermore, a significant overlap in the spatial distribution of endothelial cells and Mac6 was noted within the PVNS group (FIG. 12 Panel C), further supporting the concept of intercellular interactions between these specific subpopulations. Analysis of gene expression within the NAMPT-ITGA5-JUND signaling pathway in two ST samples revealed that NAMPT, ITGA5, and JUND were significantly upregulated in the PVNS group compared to the Control group (FIG. 12 Panel D). Therefore, the scRNA-seq research results of this invention were confirmed and expanded by spatial transcriptomics.

### Example 2. In Vivo Experiment of NAMPT Inhibitor for Treatment of PVNS

### 1. Experimental Method

### (1) Establishment of PVNS-PDX Tumor Model and Administration Regimen

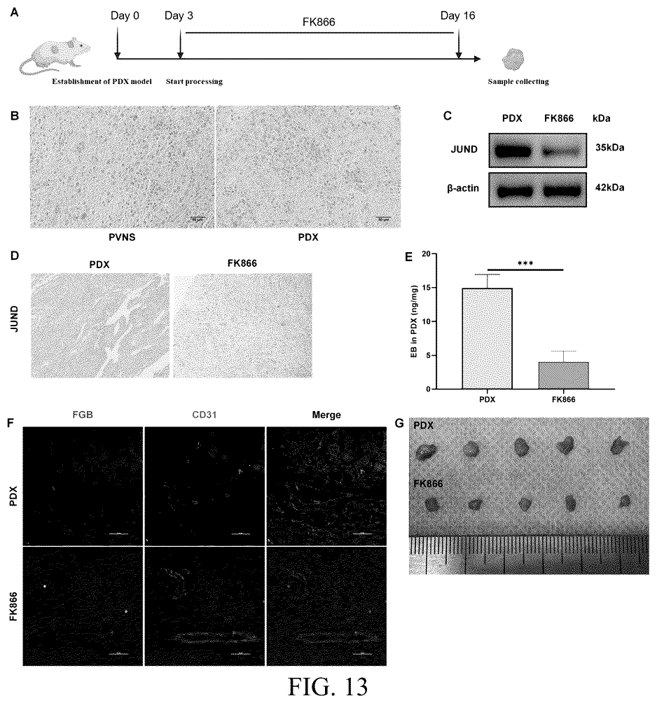

A PVNS-human-derived tissue xenograft (PDX) tumor model was established using female NOD-SCID gnotobiotic mice aged 5 to 6 weeks. The human-derived tissue was sourced from the tumor tissue of patients with a confirmed diagnosis of PVNS who underwent surgery. During the surgery, viable synovial tissue was collected and then transplanted to construct the PVNS-PDX tumor model. The mice were purchased from Vital River Laboratory Animal Technology Co., Ltd. (Beijing, China) and were housed in micro-isolator cages under specific pathogen-free conditions. Tumor grafts were prepared using sterile SmartFlow (Airtech, China). After removing the necrotic tissue, the tumor tissue was rinsed three times with physiological saline. Subsequently, the tumor mass was segmented into small pieces (diameter ranging from 1.0 to 3.0 mm) using a surgical blade. The tumor tissue (graft) was suspended in serum-free Dulbecco's Modified Eagle Medium and then cryopreserved for implantation. The surgery was carried out by using an anesthesia machine. Mice were anesthetized with 2.5% isoflurane and kept in a lateral position. The skin was sterilely prepared using surgical-grade povidone-iodine, and a small dorsal midline incision (approximately 20.0 mm) was made in the kidney area. After exposing the kidney, a tumor graft with a total volume of 25.0 mm³ was gently placed beneath the renal sac (FIG. 14 Panel A). The prepared PVNS graft was implanted within 4 hours, and the incision was closed after implantation. At the beginning of drug administration and treatment, the drug NAMPT inhibitor (FK866, HY-50876, MedChemExpress) was dissolved in 0.5% methylcellulose. Subsequently, on the third day (n=3), the aforementioned PDX mice were randomly assigned to the PDX group (i.e., the model group) and the FK866 group (i.e., the treatment group). The PDX group did not receive FK866, but was given an equal volume of physiological saline; the FK866 group was designated as the treatment group and received the drug at a dose of 30 mg/kg body weight, administered twice daily for four consecutive days, with the dosing regimen repeated weekly, for a total of two weeks; the drug was intraperitoneally injected. The mice were euthanized on day 16 after drug administration. The kidneys with tumors were carefully separated and collected for evaluation. Tumor size was measured under a microscope and calculated using the formula V=½(L +W), where L is the length and W is the width. The schematic diagram of the treatment regimen is shown in FIG. 13 Panel A.

### (2) Immunohistochemistry

The synovial tissue was fixed in 10% neutral buffered formalin for two days, followed by dehydration with a series of graded ethanol. Then, the specimen was embeded in paraffin and cut into 6 μm sections. For immunohistochemical (IHC) analysis: the paraffin sections were incubated with 3% hydrogen peroxide for 15 minutes to quench the endogenous peroxidase activity, and then incubated in 10% normal goat serum at room temperature for 1 hour to block nonspecific antigens. Subsequently, the sections were incubated overnight at 4° C. with anti-ACP5 antibody (1:100; ABclonal, A2528), CTSK (1:500; ABclonal, A1782), TNFRSF11A (1:100; ABclonal, A13382), NAMPT (1:100, ABclonal, A0256), ITGA5 (1:100, ABclonal, A19069), and JUND (1:1000, ABclonal, A5496). The next day, the sections were incubated with appropriate horseradish peroxidase (HRP)-conjugated secondary antibodies and counterstained with hematoxylin (Beyotime, C0107). All stained sections were scanned into digital images using a slide scanner for further analysis. The integrated optical density values of positive staining were evaluated using ImageJ software.

### (3) Immunofluorescence

The frozen tissue sections were thawed at room temperature for 1 hour. The 10 μm sections were rinsed with PBS, and then incubated with a blocking solution consisting of 5% serum, 2% bovine serum albumin, and 0.3% Triton X-100 in phosphate-buffered saline for 2 hours. Subsequently, the slides were incubated with primary antibodies at 4° C. overnight, rinsed with PBS, incubated with secondary antibodies for 2 hours, and then encapsulated with DAPI. The tissue sections were observed using a fluorescence microscope, and the fluorescence intensity was assessed using ImageJ. The primary antibodies used were: FGB (1:100; ABclonal, A1401) and CD31 (1:100; ABclonal, A4900).

### (4) Western Blot

Synovial tissue lysates were prepared using RIPA buffer supplemented with PMSF, incubated on ice, and physically sheared for 25 min. The proteins were extracted, and their concentration was measured using the BCA protein assay kit (Beyotime, P0011). Equal amounts of proteins (40 μg) were separated on an 8%-15% SDS-PAGE gel, transferred to polyvinylidene fluoride (PVDF) membranes, and incubated overnight with the appropriate primary antibody. Then, the PVDF membrane was washed with TBST and incubated with secondary antibodies (1:10,000; ABclonal) at room temperature for 90 min. Immunoblotting was performed using BeyoECL Plus (Beyotime), and protein bands were visualized and captured using the Tanon 2500R gel imaging system (Tanon, Shanghai, China). Band intensity was quantified using ImageJ software version 1.39 V. The primary antibodies used were: anti-NAMPT (1:1000, ABclonal, A0256); anti-ITGA5 (1:1000, ABclonal, A19069); anti-JUND (1:1000, ABclonal, A5496); and anti-β-actin (1:1000, ABclonal, AC006).

### (5) Hematoxylin-Eosin Staining

The paraffin sections were immersed in water for 5 min, and then stained with hematoxylin solution (Solarbio, Beijing, China) for 5 min. The sections were rinsed three times with distilled water, differentiated in a mixture of ammonia and acid water for 30 seconds, and then immersed in distilled water for 15 min. Subsequently, the sections were stained with eosin (Solarbio, Beijing, China) for 2 min, rinsed three times with distilled water again, dehydrated through a series of graded ethanol, and removed with xylene. Finally, these sections were mounted with neutral balsam. The tissue cavities and cell counts of each group were observed using a microscope.

### qRT-Polymerase Chain Reaction

### (6) Extraction of Total RNA from Synovial Tissue Using the Trizol Method, and Detection of RNA Concentration

HiScript III RT SuperMix for qPCR (Vazyme) was used to reverse-transcribe total RNA into cDNA. Reagents from Promega were used to synthesize cDNA from 5 μg of total RNA, followed by quantitative real-time PCR (qRT-PCR) with SYBR Green. The cDNA samples were amplified under the following conditions: 95° C. for 3 minutes, followed by 95° C. for 15 seconds and 60° C. for 15 seconds, for a total of 40 cycles. The target gene expression levels in the treatment group and PDX group were normalized and compared using the (1+e) ΔΔCT method. Primer sequences were obtained from Beijing Biotechnology Co., Ltd. Table 1 lists the primers used in the study.

TABLE 1
Primer sequences used in qRT-PCR.

| Gene name | Primer sequence (5′-3′) | SEQ ID NO: |
|---|---|---|
| NAMPT | Forward primer: | 1 |
| | ACAATATCCACCCAACACAAG | |
| | Reverse primer: | 2 |
| | TCACGGCATTCAAAGTAG | |
| ITGA5 | Forward primer: | 3 |
| | GCAAGAGCCGGATAGAGG | |
| | Reverse primer: | 4 |
| | AGGCATGGAAAGTGAGGT | |
| JUND | Forward primer: | 5 |
| | CGAACCTGTGCCCTTCC | |
| | Reverse primer: | 6 |
| | CCTGCGTGTCCATGTCG | |
| ACP5 | Forward primer: | 7 |
| | TCACCTTTGCTGAGTTCCG | |
| | Reverse primer: | 8 |
| | GCTTTCCCTCCCTCCCT | |
| CTSK | Forward primer: | 9 |
| | GAAGACCCACAGGAAGCA | |
| | Reverse primer: | 10 |
| | TGGACACCAAGAGAAGCC | |
| TNFRSF11a | Forward primer: | 11 |
| | CGCTGTGACTTGTGGACT | |
| | Reverse primer: | 12 |
| | GCCAGTGAGTGCTTGGTT | |

### (7) Vascular Permeability Assay

After 16 days of treatment, Evans Blue was used to measure the vascular permeability of mice. Evans Blue was injected into the tail artery of anesthetized mice at a dose of 20 mg/kg (dissolved in heparinized phosphate-buffered saline [100 U/ml]). 2 hours later, the tumors implanted within the renal capsule were excised, weighed, and then placed in 2 ml of formamide. The sample was incubated at 50° C. for 24 h to extract Evans blue. The concentration of Evans blue in the tissue extract was measured using a microplate reader at a wavelength of 630 nm and a standard curve of Evans blue concentration. Afterwards, the concentration was normalized and expressed in ng/mg.

### (8) Data Analysis

The statistical significance of data (mean±S.E.M.) was determined using t-tests or one-way ANOVA, followed by Tukey's post hoc test for multiple comparisons, as implemented in GraphPad Prism 5 (San Diego, USA). A p-value less than 0.05 was set as the threshold for statistical significance. Univariate statistical analysis was performed using the Wilcoxon rank-sum test and fold change (FC) to evaluate quantitative changes in the metabolomic profile. The results were visualized on a volcano plot, indicating a fold change of ≥1.5 and a p-value of <0.05. P-values were adjusted to account for multiple hypothesis testing. For confidence proteomic analysis, missing values in the data matrix were imputed as half of the minimum observed value, followed by normalization using normalization method. After log2 transformation, quantile functions in the R package “preprocess Core” were applied. Multivariate statistical analysis, including PCA, was used to observe and evaluate the overall distribution pattern of all samples.

### 2. Experimental Results

The PVNS-PDX mouse model was established in the present invention by renal capsule transplantation (FIG. 14 Panel A). Ten days after model establishment, observations showed successful implantation accompanied by significant graft vascularization (FIG. 14 Panel B). H&E staining confirmed the retention of the original tumor morphology within the graft (FIG. 13 Panel B). This proved the successful establishment of the model. Compared to the PDX group, WB analysis revealed a significant decrease in JUND expression following FK866 intervention (FIG. 13 Panel C, FIG. 14 Panel C). Similarly, IHC staining demonstrated a notable reduction in JUND expression in the FK866-treated group compared to the PDX group (FIG. 13 Panel D, FIG. 14 Panel D). To assess vascular leakage, Evan's blue dye was intravenously injected into the PDX group after 14 days. Subsequent analysis indicated a marked decrease in Evan's blue dye concentration in the FK866 group compared to the PDX group (4.040±1.618 vs. 14.93±2.003 ng Evan's blue dye/mg tissue, p<0.001) (FIG. 13 Panel E). Immunofluorescence examination revealed a significant fibrinogen (FGB) leakage around the blood vessels in the PDX group, accompanied by loosely arranged endothelial cells (CD31), with FGB accumulating in the endothelial cell gaps. In contrast, the FGB deposition around the blood vessels and the tightly arranged endothelial cells in the FK866 group were significantly reduced (FIG. 13 Panel F). Notably, 14 days of FK866 treatment significantly inhibited tumor growth, potentially due to the suppression of NAMPT expression in macrophages. As shown in FIG. 13 Panel G, the final tumor size clearly demonstrates the remarkable antitumor efficacy of FK866. Overall, FK866 treatment demonstrated significant efficacy, including improving clinical symptoms associated with the disease and inhibiting PVNS graft proliferation. This invention has discovered the effectiveness of FK866 in targeting the NAMPT-ITGA5-JUND signaling axis, proving that FK866 could alleviate endothelial cell damage and vascular permeability in the PVNS-PDX mouse model, reduce the volume of synovial tumor grafts, mitigate hemorrhagic lesions of PVNS, and hinder tumor progression.

The experimental results mentioned above indicated that NAMPT inhibitors had broad application prospects in the manufacture of medicaments for treating PVNS.

1-10. (canceled)

11. The use of NAMPT inhibitors in the manufacture of medicaments for preventing and/or treating pigmented villonodular synovitis (PVNS), and said NAMPT inhibitor is FK866.

12. The use according to claim 11, characterized in that the medicament is a preparation formed with NAMPT inhibitor as the active ingredient, in combination with pharmaceutically acceptable excipients.

13. The use according to claim 12, characterized in that the preparation is an oral preparation or an injection preparation.

14. The use according to claim 13, characterized in that the oral preparation is tablet, granule, pill, capsule, suspension, or emulsion.


SEQUENCE LISTING (12 sequences)

SEQ ID NO: 1 - DNA, 21 bp, synthetic construct
  misc_feature 1..21: note = synthesized
  source 1..21: mol_type = other DNA
acaatatccacccaacacaag

SEQ ID NO: 2 - DNA, 18 bp, synthetic construct
  misc_feature 1..18: note = synthesized
  source 1..18: mol_type = other DNA
tcacggcattcaaagtag

SEQ ID NO: 3 - DNA, 18 bp, synthetic construct
  misc_feature 1..18: note = synthesized
  source 1..18: mol_type = other DNA
gcaagagccggatagagg

SEQ ID NO: 4 - DNA, 18 bp, synthetic construct
  misc_feature 1..18: note = synthesized
  source 1..18: mol_type = other DNA
aggcatggaaagtgaggt

SEQ ID NO: 5 - DNA, 17 bp, synthetic construct
  misc_feature 1..17: note = synthesized
  source 1..17: mol_type = other DNA
cgaacctgtgcccttcc

SEQ ID NO: 6 - DNA, 17 bp, synthetic construct
  misc_feature 1..17: note = synthesized
  source 1..17: mol_type = other DNA
cctgcgtgtccatgtcg

SEQ ID NO: 7 - DNA, 19 bp, synthetic construct
  misc_feature 1..19: note = synthesized
  source 1..19: mol_type = other DNA
tcacctttgctgagttccg

SEQ ID NO: 8 - DNA, 17 bp, synthetic construct
  misc_feature 1..17: note = synthesized
  source 1..17: mol_type = other DNA
gctttccctccctccct

SEQ ID NO: 9 - DNA, 18 bp, synthetic construct
  misc_feature 1..18: note = synthesized
  source 1..18: mol_type = other DNA
gaagacccacaggaagca

SEQ ID NO: 10 - DNA, 18 bp, synthetic construct
  misc_feature 1..18: note = synthesized
  source 1..18: mol_type = other DNA
tggacaccaagagaagcc

SEQ ID NO: 11 - DNA, 18 bp, synthetic construct
  misc_feature 1..18: note = synthesized
  source 1..18: mol_type = other DNA
cgctgtgacttgtggact

SEQ ID NO: 12 - DNA, 18 bp, synthetic construct
  misc_feature 1..18: note = synthesized
  source 1..18: mol_type = other DNA
gccagtgagtgcttggtt